(19) United States
(12) Patent Application Publication
(10) Pub. No.: US 2002/0102539 A1
(43) Pub. Date: Aug. 1, 2002

(54) NUCLEOTIDE SEQUENCES AND PROTEIN SEQUENCES

(76) Inventors: ROBERT ALAN ARKOWITZ, CAMBRIDGE (GB); PETER MICHAEL ALJOSCHA NERN, CAMBRIDGE (GB)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/168,474

(22) Filed: Oct. 8, 1998

Related U.S. Application Data
(60) Provisional application No. 60/113,657, filed on Oct. 15, 1997.

(30) Foreign Application Priority Data
Oct. 8, 1997 (GB) 9721358.1
Oct. 8, 1997 (GB) 9721357.1
Jun. 12, 1998 (GB) 9812793.9

(51) Int. Cl. C12Q 1/00; C12Q 1/68; C07H 21/02; C07H 21/04; C12P 19/34

(52) U.S. Cl. 435/6; 435/91.2; 536/23.1

(57) ABSTRACT

A nucleotide sequence is described. The nucleotide sequence or the expression product of the nucleotide sequence has the capability of not substantially affecting the interaction of Gβ with Cdc24p or a hornologue thereof that is usually capable of being associated with the Cdc24p or the homologue thereof.

The present invention relates to nucleotide sequences and protein sequences. In particular, the present invention relates to nucleotide sequences and protein sequences that affeet interactions of cellular components.

According to Cerione and Zheng (The Dbl family of oncogenes Current Opinion In Cell Biology 8, 216-222 (1996)), genetic screening and biochemical studies during the past years have led to fte discovery of a certin family of cell growth regulatory proteins and oncogene products for which the Dbl oncoprotein is the prototype. Another review on Dbl is presented by Machesky and Hall (1996 Trends In Cell Biology 6 pp 3-4-310).

Cerione and Zheng (ibid) say that proto-Dbl is a 115 kDa cytoskeleton-associated protein that is found in tissues such as brain, ovary, testis and adrenal glands. Oncogenic activation of proto-Dbl occurs as a result of an amino-terminal tuncation of proto-Dbl which leaves residues 498-925 fused with the product of an as yet unidentified gene which is localised on chromosome 3.

Cerione and Zheng also say that a region located between residues 498 and 674 of proto-Dbl—which is retained by oncogenic Dbl—has significant similarities with the Saccharomyces cerevisiae cell division cycle molecule Cdc24p and the breakpoint cluster gene product Bcr (see also Hart et al 1991 Nature 354 311-314; Miyamoto et al 1991 Biochem Biophys Res Commun 181 604-610; Ron et al 1991 New Biol 3 372-379). This region—which is referred to as being the DH domain—was later shown to be responsible for the GEF (GDP-GTP Exchange factor—otherwise kmown as a guanine nucleotide exchange factor) activity of the Dbl oncoprotein and to be critical for its transforming function (see also Hart et a J Biol Chem 269 62-65).

Cerione and Zheng also report that since the initial identification of Dbl as a GEF for Rho-type GTP binding proteins, a number of oncogene products and growth regulatory Molecules have been shown to contain a DH domain in tandem with another region designated PH (i.e. a pleckstrin homology domain which is found between residues 703-812 in of proto-Dbl). Many of these products and molecules, such as Bcr, Cdc24, Sos, Vav, ect-2, Ost, Tim, Lbe, Lfc and Dbc, form a family of GEFs which have been implicated in cell growth regulation Cerione and Zheng provide details on each of these products and molecules. In addition, these and other products and molecules are discussed below.

Cerione and Zheng (ibid) end their Abstract by saying

“Despite the increasing interest in the Dbl family of proteins, there is stll a good deal to learn regarding the biochemical mechanisns that underlie their diverse biological functions.”

As mentioned above, it is known that proto-Dbl has significant similarities with the S. cerevisiae cell division cycle molecule Cdc24p which is a GEF for the Rho family GTPase molecule Cdc42p (see again Hart et al 1991 Nature A 354 311-314; Miyamoto et al 1991 Biochem Biophys Res Commun 181 604-610; Ron et al 1991 New Biol 3 372-379; Zheng et al 1994 J Biol Chem 269 2369-2372). However, whilst it is known that the Rho-family GTPases and their regulators are essential for cytoskeletal reorganisation and transcriptional activation in response to extracellular signals^{1,2}, little is known about what links these molecules to membrane receptors. For example; in the budding yeast S. cerevisiae, haploid cells respond to mating pheromone through a G-protein coupled retor (Ste2p/Ste3p) via Gβγ (Ste4p/Ste18p) resulting in cell cycle arrest, transcriptional activation, and polansed growth towards a mating partner^{4,5} Recently, the Rho-family GTPase Cdc42p and its exchange factor Cdc24p have been implicated in the maing process^{6,7} but their specific role is unknown.

However, in our. studies (which are presented below) on S. cerevisiae we have been able to identify bitherto unrecognised regions that play a key role in the interaction of cellular components. his finding has broad implications not only for the design of anti-fungal drugs (such as those that could be directed against the yeast Candida) but also in the screening and design of agents that can affect oneogenes sno as Dbl, in particular proto-Dbl.

Moreover, in our studies (which are presented below), we have identified novel cdc24 alleles which do not affect vegetative growth but drastically reduce the ability of yeast cells to mate. When exposed to mating pheromone these mutants arrest growth, activate transcription, and undergo characteristic morphological and actin cytoskeleton polarisation. However, the mutants are unable to orient towards a pheromone gient and instead position their mating projection adjacent to their.previous bud site. Strikingly, these mutants are specifically defective in the binding of Cdc24p to Gβγ. This work demonstrates that the association of a GEF and the βγ-subunit of a hetero-trirmeric G-protein (Gβγ) links receptor-mediated activaon to oriented cell growth.

The present invention also deronstrates that Far1, a cyclic dependent kinase inhibitor (CDK1) may also be implicated as being important for orientated cell growth.

Thus, according to one broad aspect of the present invention there is provided a GEF capable of interacting with a Gβ such that the interaction provides a connection between G protein coupled receptor activation and polarized cell growth.

According to another broad aspect of the present invention there is also provided an agent capable of affecting a GEF/Gβ interaction, which interaction provides a connection between G protein coupled receptor activation and polarised cell growth:

These and other aspects of the present invention are set out in the following numbered paragraphs.

1. A nucleotide sequence shown as SEQ I.D. No. 1 or a derivative, fragment, variant or homologue thereof, wherein the expression product of the nucleotide sequence has the capability of not substantally affecting the interaction of Gβ with Cdc24p or a homologue thereof that is usually capable of being associated therewith.

2. A mutant of the nucleotide sequence shown as SEQ I.D. No. 1 or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

3. A nucleotide sequence shown as SEQ I.D. No. 1 or a derivative, fragment, variant or homologue thereof or the expression product thereof for use in medicine.

4. A mutant of the nucleotide sequence shown as SEQ I.D. No. 1 or a derivative, fragment, variant or homologue thereof or the expression product thereof for use in medicine.

5. Use of a nucleotide sequence shown as SEQ I.D. No. 1 or a derivative, fragment, variant or homologue thereof or the expression product thereof in the manufacture of a medicament to affect the growth behavior of cells.

6. Use of a mutant of a nucleotide sequence shown as SEQ I.D. No. 1 or a derivative, fragment, variant or homologue thereof or the expression product thereof in the manufacture of a medicament to affect the growth behaviour of cells.

7. Use of a nucleotide sequence shown as SEQ I.D. No. 1 or a derivative, fragment, variant or homologue thereof or the expression product thereof in a screen to identify one or more agents that are capable of affecting the interaction of Cdc24p or a homologue thereof with a Gβ or an associated Rho-family GTPase.

8. Use of a mutant of a nucleotide sequence shown as SEQ I.D. No. 1 or a derivative, fragment, variant or homologue thereof or the expression product thereof in a screen to identify one or more agents that are capable of affecting the interaction of Cdc24p or a homologue thereof with a Gβ or an associated Rho-family GTPase.

9. An assay comprising contacting an agent with a nucleotide sequence shown as SEQ I.D. No. 1 or a derivative, fragment, variant or homologue thereof or the expression product thereof in the presence of a Gβ capable of being associated with Cdc24p or a homologue thereof; and determining whether the agent is capable of affecting the interaction of the nucleotide sequence or the expression product with the Gβ.

10. An assay comprising contacting an agent with a mutant of a nucleotide sequence shown as SEQ I.D. No. 1 or a derivative, fragment, variant or homologue thereof or the expression product thereof in the presence of a Gβ capable of being associated with Cdc24p or a homologue thereof; and determining whether the agent is capable of affecting the interaction of the mutant nucleotide sequence or the expression product with the Gβ.

11. A kit comprising a nucleotide sequence shown as SEQ I.D. No. 1 or a derivative, fragment, variant or homnologue thereof or the expression product thereof; and a Gβ capable of being associated with Cdc24p or a homologue thereof.

12. A kit comprising a mutant of a nucleotide sequence shown as SEQ I.D. No. 1 or a derivative, fragment, variant or homologue thereof or the expression product thereof; and a Gβ capable of being associated with Cdc24p or a homologue thereof.

13. A protein sequence shown as SEQ I.D. No. 2 or a derivative, fragment, variant or homologue thereof, wherein the protein has the capability of not substantially affecting the interaction of Gβ with Cdc24p or a homologue thereof that is usually capable of being associated with the Cdc24p or the homologue hereof.

14. A mutant of the protein sequence shown as SEQ I.D. No. 1 or a derivative, fragment, variant or homologue thereof, wherein the mutant protein has the capability of substantially affecing the interaction of Gβ with Cdc24p or a homologue thereof that is usually capable of being associated with the Cdc24p or the homologue thereof.

15. A protein sequence shown as SEQ I.D. No. 2 or a derivative, fragment, variant or homologue thereof for use in medicine.

16. A mutant of the protein sequence shown as SEQ I.D. No. 2 or a derivative, fragment, variant or homologue thereof for use in medicine.

17. Use of a protein sequence shown as SEQ I.D. No. 2 or a derivative, fragment, variant or homologue thereof in the manufacture of a medicament to affect the growth behavior of cells.

18. Use of a mutant of a protein sequence shown as SEQ I.D. No. 2 or a derivative, fragment, variant or homologue thereof in the manufacture of a medicament to affect the growth behaviour of cells.

19. Use of a protein sequence shown as SEQ I.D. No. 2 or a derivative, fragment, variant or homolegue thereof in a screen to identify one or more agents that are capable of affecting the interaction of Cdc24p or a homologue thereof thereof with a Gβ or an associated Rho-family GTPase.

20. Use of a mutant of a protein sequence shown as SEQ I.D. No. 2 or a derivative, fragment, variant or homologue thereof in a screen to identify one or more agents that are capable of affecting the interaction of Cdc24p or a homologue thereof with a Gβ or an associated Rho-family GTPase

21. An assay comprising contacting an agent with a protein sequence shown as SEQ I.D. No. 2 or a derivative, frament, variant or homologue thereof in the presence of a Gβ capable of being associated with Cdc24p or a homologue thereof; and determining whether the agent is capable of affecting the interaction of the protein sequence with the Gβ or the Rho-family GTPase.

22. An assay comprising contacting an agent with a mutant of a protein sequence shown as SEQ I.D. No. 2 or a derivative, fragment, variant or homologue thereof in the presence of Gβ capable of being associated with Cdc24p or a homologue thereof; and determining whether the agent is capable of affecting the interaction of the mutant protein sequence with the Gβ or the Rho-family GTPase.

23. A kit comprising a protein sequence shown as SEQ I.D. No. 2 or a derivative, fragment, variant or homologue thereof; and a Gβ capable of being associated with Cdc24p or a homologue thereof.

24. A kit comprising a mutant of a protein sequence shown as SEQ I.D. No. 2 or a derivative, fragment, variant or homologue thereof; and a Gβ capable of being associated with Cdc24p or a homologue thereof.

25. A (GEF capable of interacting with a Gβ such that the interaction provides a connection between G protein coupled receptor activation and polarised cell growth.

26. An agent capable of affecting a GEF/Gβ interaction, which interaction provides a connection between protein coupled receptor activation and polarised cell growth.

27. A sequence selected from: SEQ ID No. 15 or SEQ ID No. 16 or SEQ ID No. 17 or SEQ ID No. 18 or SEQ ID No. 19.

28. An assay method comprising the use of the sequence presented in paragraph 28 or a nucleotide sequence coding for same.

29. Use of an agent identified by the assay of paragraph 9 or paragraph 10 or paragraph 21 or paragraph 22 or paragraph 28 in the manufacture of a medicament which affects cell growth.

By way of example in a broad aspect, the present invention provides a nucleotide sequence shown as SEQ I.D. No. 1 or a derivative, fragment variant or homologue thereof, wherein the expression product of the nucleotide sequence has the capability of not substantially affecting the interaction of Gβ with GEF or a homologue thereof that is usually capable of being associated therewith.

The term “expression product of the nucleotide sequence has the capability of not substantially affecting the interaction of Gβ with GEF or a homologue thereof that is usually capable of being associated therewith” means that if the expression product were to be present within GEF and tile GEF were to be contacted with Gβ then the expression product would not substantially affect the interaction of Gβ with GEF.

Thus, alternatively expressed, the present invention covers a nucleotide sequence shown as SEQ I.D. No. 1 or a derivative, fragment, variant or homologue thereof, wherein the expression product of the nucleotide sequence has the capability of not substantially affecting the interaction of Gβ with GEF or a homologue thereof that is usually capable of being associated therewith if the expression product were to be present within GEF and the GEF were to be contacted with Gβ.

With this aspect of the present invention, the expression product need not necessarily be present within GEF and/or the GEF need not necessarily be contacted with Gβ. By way of example, the expression product can be part of a truncated GEF and/or part of a fused protein. However, if the expression product were present within GEF, then preihrably the GEF is not in its natural environment. By way of example, the GEF can be in an isolated form—such as in an assay device. Likewise, if the expression product were contacted with Gβ then preferably the Gβ is not in its natural environment. By way of example, the Gβ can be in an isolated form—such as in an assay device.

The present invention also covers a mutant of the nucleotide sequence shown as SEQ I.D. No. 1 or a derivative, fragment, variant or homologue thereof, wherein the expression product of the mutant nucleotide sequence has the capability of substantially affectin the interaction of Gβ with GEF or a homologue thereof that is usually capable of being associated therewith.

The term “expression product of the mutant nucleotide sequence has. the capability of substantially affecting the interaction of Cip with GEF or a homologue thereof that is usually capable of being associated therewith” means that if the expression product were to be present within a GEF like entity (such as GEF bearing that mutation) and that GEF like entity were to be contacted with Gβ then the expression product would substantially affect the interaction of Gβ with that GEF like entity.

Thus, alternatively expressed, the present invention also covers a mutan of the nucleotide sequence shown as SEQ I.D. No. 1 or a derivative, fragment, variant or homologue thereof, wherein the expression product of the mutant nucleotide sequence has the capability of substantially affecting the interacon of Gβ with GEF or a homologue. thereof that is usually capable of being associated therewith if the expression product were to be present within GEF and the GEF were to be contacted with Gβ.

With this aspect of the present invention, the expression product need not necessarily be present within the GEF like entity and/or the GEF like entity need not necessarily be contacted with Gβ.By way of example, the expression product can be part of a truncated GEF and/or part of a fused protein. The GEF like entity may be in an isolated form—such as in an assay device. Likewise, if the expression product were contacted with Gβ then preferably the Gβ is not in its natural environment. By way of example, the Gβ can be in a isolated form—such as in an assay device.

In one preferred aspect, the GEF is Cdc24p. Other suitable GEFs have been mentioned above.

Thus, the present invention also covers in a broad aspect a nucleotide sequence shown as SEQ I.D. No. 1 or a dervative, fragment, variant or homologous thereof wherein he expression product of the nucleotide sequence has the capability of not substantially affecting the interaction of Gβ with Cdc24p or a homologue thereof that is usually capable of being associated therewith.

The term “expression product of the nucleotide sequence has the capability of not substantially affecting the interaction of Gβ with Cdc24p or a homologue thereof that is usually capable of being associated therewith” means that if the expression product were to be present within Cdc24p and the Cdc24p were to be contacted with Gβ then the expression product would not substantially affecting the interaction of Gβ with Cdc24p.

Thus, alternatively expressed, the present invention covers in a broad aspect a nucleotide sequence shown as SEQ I.D. No. 1 or a derivative, fragment, variant or homologue thereof, wherein the expression product of the nucleotide sequence has the capability of not substantially affecting the interaction of Gβ with Cdc24p or a homologue thereof that is usually capable of being associated therewith if the expression product were to be present within Cdc24p and the Cdc24p were to be contacted wit Gβ.

With this aspect of the present invention, the expression product need not necessarily be present within Cdc24p and/or the Cdc24p need not necessarily be contacted with Gβ. By way of example, the expression product can be part of a truncated Cdc24p and/or part of a fused protein. However, if the expression product is present within Cdc24p, then preferably the. Cdc24p is not in its natural environment By way of example, the Cdc24p can be in an isolated form—such as in an assay device. Likewise, if the expression product were contacted with Gβ then preferably the Gβ is not in its natural environment. By way of example, the Gβ can be in an isolated form—such as in an assay device.

By way of further example, the present invention also covers a mutant of the nucleotide sequence shown as SEQ I.D., No. 1 or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

The tenn “expression product of the mutant nucleotide sequence has the capability of substantially affecting the interaction of Gβ with Cdc24p or a homologue thereof that is usually capable of being associated therewith” means that if the expression product were to be present within a Cdc24p like entity (such as Cdc24p bearing that mutation) and that Cdc24p like entity were to be contacted with Gβ then the expression product would substantially affect the interaction of Gβ with hat Cdc24p like entity.

With this aspect of the present invention, the expression product need not necessarily be present within Cdc24p and/or the Cdc24p need not necessarily be contacted with Gβ. By way of example, the expression product can be part of a truncated Cdc24p and/or part of a fused protein. The Cdc24p like entity may be in an isolated form—such as an assay device. Likewise, if the expression product were contacted with Gβ then preferably the Gβ is not in its natural environment. By way of example, the Gβ can be in an isolated form—such as in an assay device.

In a preferred aspect, the present invention covers the sequences of the present invention in isolated form-in other words the sequences are not in their natural environment and when they have been expressed by their natural coding sequences which are under the control of their natural expression regulatory elements (such as the natural promoter etc.). By way of example the sequences may be in an assay device.

It is to be noted that the nucleotide sequence presented as SEQ ID No. 1 is quite different to the DH domain and the PH domain discussed by Cerione and Zheng (ibid). It is also to be noted that the nucleotide sequence presented as SEQ ID No. 1 is in a region quite different to the DH domain and the PH domain.

One important aspect of the present invention is hat we have found it is possible to affect the interaction of Cdc24p with a β submit (such as Ste4p) or even a βγ subunit (such as Ste4p/Ste18p) of a hetero-trimeric G-protein (hereinafter collectively referred to as “Gβ”). If the interaction is detrimentally affected (such as lost) then this may in turn prevent (or at least reduce) signaling (possibly GEF activity) being passed to the the Rho-family GTPase (Cdc42p). Hence, the present invention also covers the use of any one or more of the aforementioned aspects of the present invention to have an effect on a signal being passed to the Rho-family GTPases.

The term “derivative, fragment, variant or homologues” in relation to the protein Sequence ID ID No. 1 of the present invention includes any substitution of, modification of, replacement of, deletion of or addition of one (or more) nucleic acid from or to the sequence providing the resultant nucleotide sequence or the expression product thereof has the capability of not substantially affecting the interaction of Gβ with Cdc24p or a homologue thereof that is usually capable of being associated with the Cdc24p or the homologue thereof. In partcular, the term “homologue” covers homology with respect to function. With respect to sequence homology (i.e. sinilarity), prefrably there is a least 75%, more preferably at last 85%, more preferably at least 90% homology to the sequence shown as SEQ ID No. 1 inte attached sequence listings. More preferably there is at least 95%, such as at least 98%, homology to the sequence shown as SEQ ID No. 1 in the attached sequence listings.

The term “derivative, fragment, variant or homologue” in relation to the protein Sequence ID No. 2 of the present invention includes any substitution of, modification of, replacement of, deletion of or addition of one (or more) amino acid from or to the sequence providing the resultant amino acid sequence has the capability of not substantially affecting the interaction of Gβ with Cdc24p or a homologue thereof that is usually capable of being associated with the Cdc24p or the homologue thereof In particular, the term “homologue” covers homology with respect to function. With respect to sequence homology (i.e. similarity), preferably there is at least 75%, more preferably at least 85%, more preferably at least 90% homology to the sequence shown as SEQ ID No. 2 in the attached sequence listings. More preferably there is at least 95%, such as at least 98%, homology to the sequence shown as SEQ ID No. 2 in the attached sequence listings.

An example of a Eminent of the expression product of SEQ ID No. 1 that has the capability of not substantially affecting the interaction of Gβ with Cdc24p or a homologue thereof that is usually capable of being associated with the Cdc24p or the homologue thereof is the amino acid sequence presented as SEQ ID No. 15 or SEQ ID No. 16. The present invention also covers nucleotide sequences coding for such sequences.

With respect to the mutated sequences then, in a preferred aspect, the mutated. sequence comprises one or more mutations in the region presented as SEQ ID No. 15 or SEQ ID No. 16.

An example of a fragment of the expression product of a mutant SEQ ID No. 1 that has the capability of substantially affecting the interaction of Gβ with Cdc24p or a homologue thereof that is usually capable of being associated with the Cdc24p or the homologue thereof is the amino acid sequence presented as SEQ ID No. 17 or SEQ ID No. 18 or SEQ ID No. 19. The present invention also covers nucleotide sequences coding for such sequences.

In particular, the term “homology” as used herein may be equated with the term “identify”. Relative sequence homology (i.e. sequence identity) can be determined by commercially available computer programs that can calculate % homologue between two or more sequences. Typical examples of such computer programs are BLAST and CLUSTAL.

Sequence homology (or identity) may moreover be determined using any suitable homology algorithm, using for example default parameters. Advantageously, the BLAST algorithm is employed, with parameters set to default values. The BLAST algorithm is described in detail at http://www.ncbi.nih;gov/BLAST/blast_help.htl, which is incorporated herein by reference. The search parameters are defined as follows, and. are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Kailin and Altschul (see. http://www.ncbi.nih.gov/BLAST/blast_help.html) with a few enhancements The BLAST programs were tailored for sequence similarity searching, for example to identify homologues to a query sequence. The programs are not generally useful for motif-style searching. For a discussion of basic issues in similarity searching of sequence databases, see Altschul et al (1994) Nature Genetics 6:119-129.

The five. BLAST programs available at http:I/www.ncbi.nlm.nih.gov perform the following tasks:

blastp compares an amino acid query sequence against a protein sequence database,

blastn compares a nucleotide query sequence against a nucleotide sequence database;

blastx compares the six-frame conceptual translation products of a nucleotide query sequence Roth strands) against a protein sequence database;

tblastn compares a protein query sequence against a nucleotide sequence database dynamically translated in all six reading frames (both strands). tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIOnucleotide sequence Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by. chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to temn is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250and IDIENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU prograrn of Claverie & States (1993) Computers and Chemistry 17:191-201, or, for BLASTN, by the DUST program of Tatusov and Lipman (see http://www.ncbi.nlm.nih.gov). Filtering can ellimnate statistically significant but biologically uninteresting reports from the blast output (e.g., hits against common acidic-, basic-or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”)

Filtering is only applied to the query sequence (or its translation products), not to database sequences. Default filtering is DUST for BLASTN, SEQ for other programs.

It is not unusual for nothing at all to be masked by SEQ, XNU, or both, when applied to sequences in SWISS-PROT, so filtering should not be expected to. always yield an effect.

Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST.

Other computer program methods to determine identify and similarity between the two sequences include but are not limited to the GCG program package (Devereux et al 1984 Nucleic Acids Research 12: 387and FASTA (Atschul et al 1990 J Molec Biol 403-410).

The term “variant” also encompasses sequences that are complementary to sequences that are capable of hymidising to the nucleotide sequences presented herein.

Preferably, the term “variant” encompasses sequences that are complementary to sequences that are capable of hydnidising under stringent conditions (eg 65° C. and 0.1×SSC {1×SSC=0.15 M NaCl, 0.015 Na₃ citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences at can hybridise to the nucleotide sequences of the present invention (including complementary sequences of those presented herein).

The term “hybridization” as used herein shall include “the process by which a strand of nucleic acid joins with a complementary strand trough base pairing” (Coombs J (1994) Dictionary of Biotechology, Stockton Press, New York, N.Y.) as well as the process of amplification as carried out in polymerase chain reaction techlologies as described in Dieffenbach C W and G S Dveksler (1995, PCR Primer, a Laboratory Manual, Cold Spring Harbor Press, Plainview, N.Y.).

Also included within the scope of the present invention are polynucleotide sequences that are capable of hybridizing to the nucleotide sequence of the present invention or other nucleotide sequences coding for the protein sequence of the present invention under conditions of intermediate to maximal stringency. Hybridization conditions are based on the melting temperature (Tm) of tie nucleic acid binding complex, as taught in Berger and Kimmel (1987, Guide. to Molecular Cloning Techniques, Methods in Enzymology, Vol 152, Academic Press, San Diego, Calif.), and confer a defied “stringency” as explained below.

Maximum stringency typically occurs at about Tm-5° C. (5° C. below the Tm of the probe); high strigency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC).

Examples of homologues of Cdc24p include but are not limited to any one or more of the homologues listed above or below, such as proto-Dbl, Bcr, Sos, Vav, ect-2, Ost, Tim, Lbc, Lfc and Dbc.

The term “mutant” in relation to the nucleotide sequence of the present invention means a variant of SEQ ID No. 1 but wherein that variant or the expression product thereof has the

capability of substantially affecting the interaction of Gβ with Cdc24p or a homologue thereof that is usually capable of being associated with the Cdc24p or the homologue thereof.

Preferred mutants of the nucleotide sequence of the present invention include any one or more of the nucleotide sequences presented as SEQ ID No. 3, SEQ ID No. 5 or SEQ ID No. 7.

The term “mutant” in relation to the protein sequence of the present invention means a variant of SEQ ID No. 2 but wherein that variant has the capability of substantially affecting the interaction of Gβ with Cdc24p or a homologue thereof that is usually capable of being associated with the Cdc24p or the homologue hereof

Preferred mutants of the protein sequence of the present invention include any one or more of the protein sequences presented as SEQ ID No. 4, SEQ ID No. 6 or SEQ ID No. 8.

The term “growth behaviour” includes growth per se (but not vegetative growth of yeast), growth control and growth orientation of cells. In some aspects, it includes at least growth orientation of cells. The term may also include the mating pattern (e.g. mating per se or mating behaviour) of cells.

For a preferred aspect of the present invention, any one or more of the nucleotide sequence of the present invention or the expression product thereof, or the mutant nucleotide sequence of the present invention or the expression product thereof, or the protein of the present invention, or the mutant protein of the present invention may be within a tiusgenic organism or cell (such as being an integral part thereof)—that is an organism or cell that is not a naturally occurring organism or cell and wherein the organism or cell has been prepared by use of recombinant DNA techniques. The transgenic cell may be pat of or contained within tissue.

Preferably, the transgenic organism or cell is a yeast, an animal (such as a mammal) or an animal cell (such as a mammalian cell).

In preferred embodiments, the transgenic organism is a transgenic yeast or a transgenic mouse.

Transgenic yeast may be prepared by appropriately adapting the teachings of Ito et at Journal of Bacteriology 153 163-168; Rose et al 1991 Methods in yeast. genetics: a laboratory course manual Cold Spring Harbor, N.Y: Cold Spring Harbor Press).

Transgenic mammals or mammalian cells may be prepared by appropriately adapting the teachings of Ausubel et al 1992 Short Protocols in Molecular Biology 2nd Ed. New York: John Wiley and Sons).

The transgenic organism or transgenic cell of the present invention therefore provides a simple assay system that can be used to determine whether one or more agents (e.g. compounds or compositions) have one or more beneficial properties. By way of example, the assay system of the present invention may utilise a mating phenotype and/or the assay system may be a two-hybrid interaction assay.

By way of example, if the transgenic organism is a transgenic yeast which comprises the nucleotide sequence presented as SEQ ID No. 1 or the expression product thereof (namely the protein sequence presented as SEQ ID No. 2) then the yeast could be used to screen for agents that bind to this nucleotide sequence or the expression product thereof and in doing so affect the growth behaviour of the yeast. If an agent produces such a detrimental effect (such as drastically reducing the ability of the yeast to mate), then that agent may also affect the interaction of Gβ with Cdc24p or another Cdc24p entity that is usually capable of being associated therewith. This aspect of the present invention could allow workers to screen for anti-fungal agents, such as agents that could be used to treat or combat Candida.

By way of further example, if the transgenic organism is a transgenic yeast which comprises the nucleotide sequence presented as SEQ ID No. 1 or the expression product thereof then the yeast could be used to screen for agents that bind to his nucleotide sequence or expression product thereof and in doing so affect the growth behaviour of the yeast. If an agent produces a detrimental affect (such as drastically reducing the ability of the yeast to mate), then that agent is likely to detrimentally affect the interaction of Gβ with a homologue of Cdc24p with which it is usually capable of being associated. This could allow workers to screen for compounds or compositions that could for example influence the in vivo expression or behaviour of effect of proto-oncogenes and the like—such as proto-Dbl.

By way of further example, if the transgenic organism is a transgenic yeast which comprises a mutant of the nucleotide sequence in accordance with the present invention then the yeast could be used to screen for agents that affect the growth behaviour of the yeast. If an agent produces a marked affect—such as restoration to a normal growth behaviour or a further detrimental growth behaviour—then workers could screen for compounds or compositions that could for example influence the in vivo expression or behaviour or effect or activity of a Cdc24 homologue, such as, but not limited to proto-oncogenes such as, Dbl and/or Vav.

By way of further example, if the transgenic organism is a transgenic:yeast which comprises a homologue (e.g. Dbl) of the nucleotide sequence shown as SEQ ID No. 1 or an expression product thereof then workers could see if that homologue or the expression product thereof had an effect on the growth behaviour of yeast, and thus also to see if it had an effect on the interaction of Gβ with a homologue of Cdc2p. In addition, workers could use those transgenic yeast to screen for agents that modified the effect—such as enhance the growth behaviour or detrimentally affect the growth behaviour. In this aspect, agents that affect the growth behaviour may also influence the activity of oncogenes (or even parts thereof) and therefore have potential as therapeutic agents.

The assays of the present invention may also be used to screen for agents that affect the interaction of Cdc24p or a Cdc24p homologue with Gβ to determine whether that effect has a downstream effect on a Rho-family GTPase.

For example, with the present invention—such as by use of the assays of the present invention-it is possible to devise and/or to screen for peptide inhibitors which block GEF/Gβ interaction. In this regard, peptides and peptidyl derivatives based regions encompassing mutants may be used to block and/or antagonise GEF (such as the ptoto-oncogenes Dbl or Vav) Gβ interaction. Derivadves of these peptides (including peptide mimics) which bind with higher afiity may also be used-The perturbation of these interactions may be of therapeutic value for example in treatment of cancers.

In addition, by use of the present invention it is possible to devise simple yeast. based assay systems (utilising mating function and interaction reporters). These assay synods will be extremely useful for high. through-put screening to identify molecules perturbing the GEF/β interaction.

In addition, it is possible to devise and/or screen for agents that can modulate (e.g interact), preferably selectively modulate (interact),with and affect Cdc24p/β interactions. Hence, it would be possible to devise and/or to screen for anti-fungal agents directed at invasive and/or pathogenic yeasts such as, but not limited to Candida albicans and/or Cryptococcus neoformans.

If the assay of the present invention utilises a transgenic organism amording to the present invention then transgenic organism may comprise nucleotide sequences etc. that are additional to the nucleotide sequences of the present invention in order to maintain the viability of the transgenic organism.

In the assays of the present invention, the agent can be any suitable compound, composition as well as being (or even including) a nucleotide sequence of interest or the expression product thereof Hence, if any one of the nucleotide sequences of the present invention are contained within a transgenic organism—such as a transgenic yeast—then that transgenic organism may also contain that nucleotide sequence of interest. If the agent is a nucleotide sequence, then the agent may be, for example, nucleotide sequences from organisms (e.g. higher organisms—such as eukaryotes) that restore or increase the growth behaviour. Agents which affect the growth behaviour may also influence the activity of homologous oncogenes and may therefore be potential therapeutic agents.

The following samples were deposited in accordance with the Budapest Treaty at the recognised depositary of The National Collections of Industrial and Marine Bacteria Limited (NCIMB) at 23 St. Machar Drive, Aberdeen, Scotland, United Kingdom, AB2 IRY on Oct. 3, 1997:

E.coli CMK603 PRS414CDC24 (WT)-Deposit Number NCIMB 40898

E.coli CMK603 PRS414CDC24 (M1)-Deposit Number NCIMB 40899

E.coli CMK603 PRS414CDC24 (M2)-Deposit Number NCIMB 40900

E.coli CMK603 PRS414CDC24 (M3)-Deposit Number NCIMB 40901

Deposit NCIMB 40898 is in respect of cdc24 (wt); Deposit NCIMB 40899 is in respect of cdc24-m1; Deposit NCIMB 40900 is in respect of cdc24-m2 Deposit NCIMB 40901 is in respect of cdc24-m3.

In accordance with a preferred aspect of the present invention, the nucleotide sequence is obtainable from, or the protein is expressable from the nucleotide sequence contained within the respective deposit. By way of example, the respective nucleotide sequence may be isolated from the respective deposit by use of appropriate restriction enzymes or by use of PCR techniques.

The present invention will now be described only by way of example, in which reference is made to the following Figures:

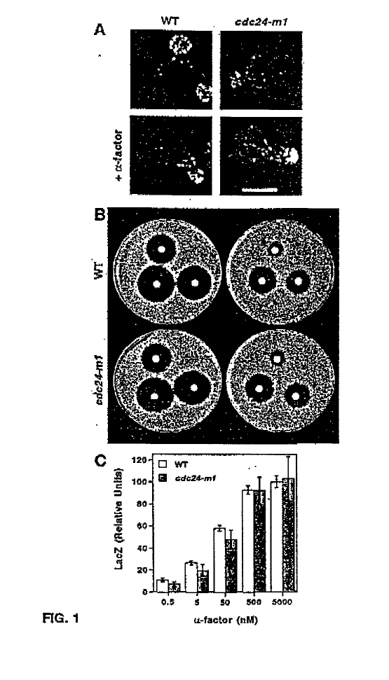

FIG. 1 which presents some photographs and a graph;

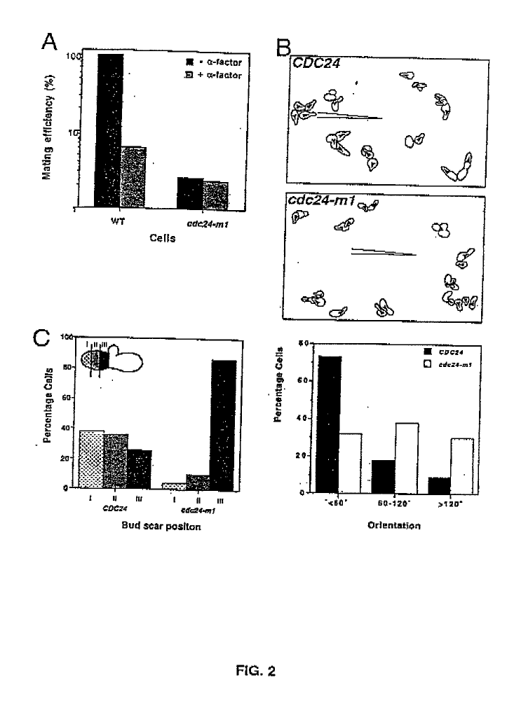

FIG. 2 which presents some images and graphs;

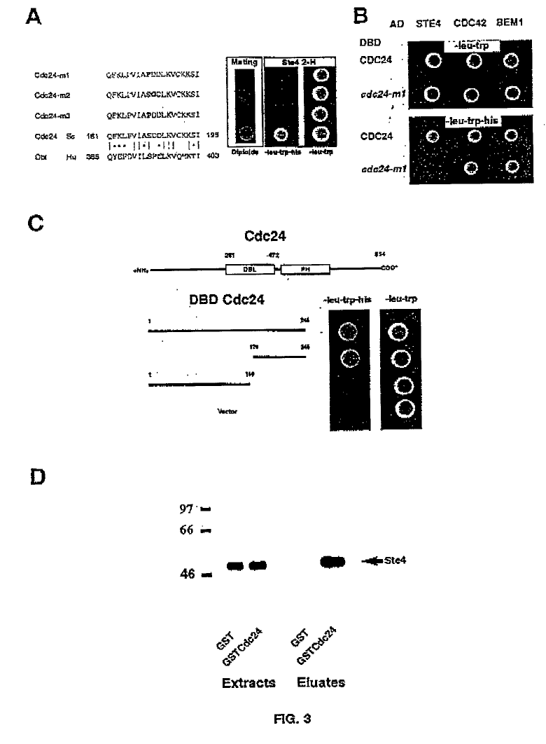

FIG. 3 which presents some photographs, a sequence, and a pictorial representation of Cdc24 and DBD Cdc24; and

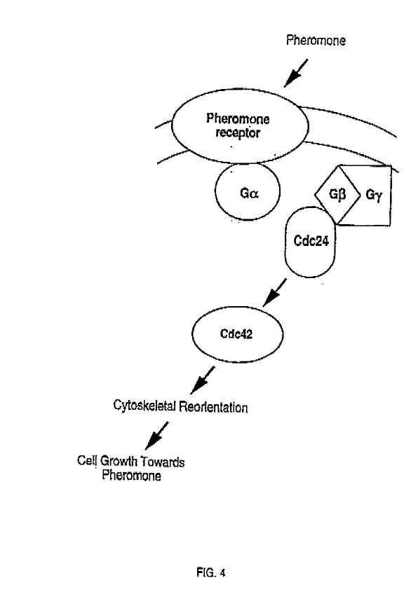

FIG. 4 which presents a pictorial representation of a cellular interaction.

The Figures are discussed in more detail later on.

### Materials and Methods

General techniques

Strains were constructed using standard techniques²¹. All constructs were verified by DNA dye terminator cycle sequencing (ABI377 sequencer).

Strains

pRS414CDC24 contains the CDC24 ORF including 258 bp upstream of ATG.

Oligonucleotide-directed mutagenesis was used to introduce silent base changes that resulted in the following ten new restriction sites in CDC24: NheI (bp-12), KasI (bp 283), AatII (bp 681), PstI (bp 1207), RsrII (bp 1369), BstEII (bp 1426), XhoI (bp 1758), MluI (bp 1963), SalI (bp 2061), BamHI (bp 2485). RAY410 (MATa, leu2, CDC24::LEU2, ade2, lys2, his3, trp1, ura3, pEG(KT)CDC24) was derived from the diploid YOC380²² which was tansformed with pEG(KT)CDC24²³ and sporulated, RAY950 is isogenic to RAY410 but has pRS416GalHis₆CDC24 as a rescuing plasmid. RAY928 (MATa, leu2-3, 112, ura3-52, his 3-D200, trp1-D901, lys 2-801, suc2-D9, CDC24::HIS5 pEG[KT]CDC24) and RAY931 (same as RAY928 but MATa ade2, LYS2) were made by transformation of SEY6210 and 6211 with pEG(KT)CDC24 followed by PCR-based gene disruption of CDC24. The CDC24 ORF was replaced with S. pombe HIS5²⁴, flanked by LoxP sites. Replacement of CDC24 in SEY6211 with a PCR-generated integration cassette consisting of TRPl fused to 343 bp of CDC24 promoter followed by 1704 bp of CDC24 or cdc24-m1 ORF was used to construct RAY1034 or RAY1035, respectively.

### IDENTIFICATION OF cdc24 MUTANTS WITH SPECIFIC DEFECTS IN CELL MATING

A) Construction of a Library of cdc24 Random Mutants

Error-prone PCR was used to generate a library of cdc24 mutants in a plasmid vector suitable for phenotypic screening in yeast.

1) Plasmid

pRS414 CDC24 with upstream region and new restriction sites (refe to as pRS414CDC24).

2) Mutagec PCRs

Conditions from Fromant, M. , Blanquet, S. & Plateau, P. Direct random mutagenesis of gene-sized DNA fragments using polymerase chain-reaction. Analytical Biochemisty 224, 347-353 (1995)

Different PCR-conditions were tested and the error-rate was determined by DNA sequencing. The following conditions were used for constructing the library used in the screen.

Composition of PCR-reactions (25 μl each):

| DNA pRS414CDC24 600pM | |
|---|---|
| dATP | 0.23 mM |
| dCTP | 0.20 mM |
| dTTP | 2.9 mM |
| dGTP | 0.42 mM |
| Buffer | PCR Buffer supplied with Taq-polymerase |
| MgCl₂ | 4 mM |
| MnCl2 | 0.5 mM |
| Taq (Ampli-Taq) | 2 U per reaction |
| Primer: | ˜0.5 mM |
| PCR-cycles: | |
| step 1 94° C. | 5 min |
| step 2 91° C. | 1 min |
| step 3 51° C. | 1 min |
| step 4 72° C. | 3 min |
| step 5 72° C. | 5 min |
| step 6 4° C. | pause |

16 cycles (steps 2-4)

3) Library construction:

The PCR products were digested with AafII and NheI (680 bp corresponding to amino acid 1 -227) were mutagenised and the resulting fragment ligated into pRS414CDC24 (cut with the same enzymes). Ligations were tansformed into E. coli by electroporation and >50,000 transformants pooled for plasmid isolation.

B) Phenotypic Screening for Cell-Mating Specific cdc24 Alleles

Rationale:

To identify mutant cdc24 alleles which cause defects in cell mating but allow vegetative growth. Yeast strain RAY950, in which expression of CDC24 is repressed in glucose medium, was used.

1) Library plasmids were transformed into RAY950 and transformants selected on SC-trp plates which contained 2% glucose. As RAY950 does not grow on glucose plates this procedure eliminated all non-functional cdc24 mutants.

2) Transfomants were replica-plated onto a lawn of WT (screen 1) or Δfus1 Δfus2 (screen 2) tester cells, incubated at 30° C. for 3 hrs and replica-plated onto plates selecting for diploids or RAY950 derived haploids. Mating defective mutants were identified by comparing the pattern of colonies on the two sets of plates and candidate mutants were picked from the original transformation plates for retesting.

3) Plasmids from mutants were isolated by transformation into E. coli Isolated plasmids were retransformed into RAY950, RAY928 and RAY931 for independent confirmation of phenotype and retested for defects in cell mating.

4) Mutations of confirmed mutants were identified by DNA sequencing. Multiple mutations were separated by subcloning and the mutation responsible for the phenotype identified by mating tests in RAY950.

5) A total of ˜5,000 yeast transformants were tested in each screen

Screen 1 identified two mutants (cdc24-m1, cdc24-m2).

Screen 2 identified one mutant (cdc24-m3).

Phenotypic analyses

Quantitative matings¹⁰, matings in the presence of saturating pheromone13, halo-assays²⁶ using sstl:: URA3 strains, and Fussllacz measurements with pSG231¹¹ were carried out as described. Halo assays showed MATa and MATa cdc24-m1 cells secreted α-factor and α-factor, respectively. Actin was visualised with rhodatmine phaloidin²⁷ on a Biorad-MRC600 confocal microscope and pictures are projections of 4-6 0.5 mm z-series steps. For α-factor treatment, cells were incubated with 5 mM α-factor for 2 hr. RAY1034 and RAY1035 cells were used to determine bud scar positions on zygotesI⁴ visualised with Calcoflour⁸. Similar results were observed with the position of the bud scar on shmoos. Direct measurement of cell orientation in a pheromone gradient was carried out essentially as described¹². A pheromone gradient was generated using a micropipet filled-with 80 mM a-factor injected at 105 kPa into 1 ml of YEPD media layered on top of cells embedded in 2% Low Melting Point (LMP) agarose. Cells shape was recorded by video microscopy On a heated stage at 35°for 4-7 hr and data analysis was from traced cell outlines¹⁴ Mating projections were formed at the same pheromone concentrations and budding, that is non-responding cells were seen at similar distances from the micropipet in both strains.

Two-Hybrid methods

STE4, BEM1 (372-551 aa), CDC42[C178S], and CDC24 Icdc24-m1 (1-288, 1-160, and 170-245 aa) were cloned by PCR into pGAD424 (AD, GAL4 activation domain) or pAS1 (DBD, GAL4 DNA binding domain). Plasmids were tansformed into HF7c. For determination of STE18 requirement, PCR-based gene disruption was carried out in PJ69-4A (MATa, trp1-901, leu2-3,112, ura-3-52, his3-200, gal4D, gal80D, GAL2-ADE2, LYS2::GAL1-HIS3, met2::GAL7-lacZ)²⁹, replacing the entre STE18 ORF with K Lactzs URA3³⁰. For all two-hybrid experiments, equal amounts of transmformants were spotted on SC-leu-trp and SC-leu-trp-his plates, identical results were obtained with at least four transformants, and for Dste18 two independent deletion strains. All strains for two-hybrid analyses expressed similar amounts of AD-and DBD-fusion proteins of the expected sizes, as determined by SDS-PAGE and immuno-blotting. None of the DBD fusions showed any self-activation using two different non-interacting AD fusions.

In vitro binding studies

A fragment of CDC24 (1-472 aa) in pGEX-2T (Pharmacia) and His₆Ste4p (PTrcSte4) were expressed in E. coli. Cells were resuspended in buffer A (PBS, 0.1% TX-100,Phenyl Methyl Sulfonyl Fluoride (PMSF), leupeptin, chymostatin, pepstatin, aprotinin) and lysed by snap freezing in liquid nitrogen followed by sonicatiom Insoluble material was removed by centrifugation (10,000 g). Mixed supernatants (denoted cell extracts) containing His₆Ste4 and GSTCdc24 fusions were incubated with GSH-agarose (Sigma Chemical Co. ) at 4° for 1 hr.

Resin was washed 3 times with buffer A. Resin samples (referred to as eluates) and extracts were analyzed by SDS-PAGE, immuno-blotting probed with Omni-probe anti-sera (Santa Cruz), and visualised with enhanced chemiluminescence (Amersham). GSTCdc24p (1-127 aa), similar to GST, did not bind His₆Ste4p. Similar results were observed in 5 independent experiments.

C) Ste4p Mutants

Ste4p is the β-subunit of the heterodimeric G protein that can usually associate with Cdc24p exemplified by nucleotide SEQ ID No. 9 and amino acid SEQ ID No. 10. A mutation in STE4 exemplified by nucleotide SEQ ID No. 11 and SEQ ID No. 13 and amndo acid SEQ ID No. 12 and SEQ ID No. 14 prevented the interaction of the mutant G protein β subunit with Cdc24p. Thus, it is possible to devise assays based on this mutation to screen for agents capable of modifying the non-interactive behaviour of the mutant G protein βsubunit with Cdc24p. In addition, the assay could be used to study Cdc24p homologues or even Cdc24p derivatives or homologues to see if those derivatives or homologues affect the non-interactive behaviour of the mutant G protein, βsubunit.

The Ste4p mutants are also aspects of the present invention.

In this regard, the present invention also covers an STE4 mutant.

The present invention also covers a mutation of the β-subunit of the heteroditneric G-protein that can usually associate with GEF (preferably Cdc24p) that is capable of preventing the interaction of the mutant G protein subunit with GEF (preferably Cdc24p).

Hence, a further aspect of the present invention is a mutation in STE4-i. e. on the β-subunit of the heterodimeric G protein that can usually associate with Cdc24p. This mutation prevents At interaction of the mutant G protein subunit with Cdc24p. Thus, likewise,. it is possible to devise similar assays based on this mutation to screen for agents that modify the non-interactive behaviour of the mutant G protein with Cdc24p. In addition, the assay could be used to study Cdc24p homologues or even Cdc24p derivatives or variants to see if those derivatives or variants affect the non-interactive behaviour of the mutant G protein. The sequences associated with this aspect of the present invention are shown as SEQ ID No. 9 etc. The present invention also covers variants or derivatives of such sequences—wherein the variants or derivatives of the wildtype sequences do not substantially affect Cdc24 interaction; and wherein the variants or derivatives of the mutant sequences do substantially affect Cdc24 interaction.

D) Assay System to Monitor the Effects of Two Human Oncogenic Agents on an S. Cerevisiae Yeast Mutant With a Mating Defect.

An assay system was devised to establish whether two different proto-oncogenes could complement the S. cerevisiae yeast phenotype (cdc24-m1) mating defect as described above and in Nern and Arkowitz (Nature (1998) 391: 195-198). The two oncogenic agents used. were the human proto-oncogene, proto-Dbl and the mouse C4 protein which is almost identical to the human sequence, C5 Vav, and which is referred to hereafter as Vav. The S. cerevisiae cell division cycle molecule, Cdc24p, which is a protein with similarities to proto-Dbl was used as a positive control in addition to the Cdc24p of the related yeast K. lactis.

Transgenic yeast organisms which co-expressed the nucleotide. sequence (SEQ ID No. 3) for the cdc24-m1 mating defect and the nucleotide sequence of interest (NOI) encoding either proto-Dbl, Vav or two related Cdc24p's were used.

The expression levels of the proto-oncogene, proto-Dbl, in S. cerevisiae were relatively low compared with the expression levels of the Cdc24p protein from either S. cerevisiae or K. lactis.

Qualitatively, both proto-Dbl and K. lactis Cdc24 proteins partially complemented the mating defect in the cdc24-m1 mutant. This result is in contrast to that obtained with the oncogenic form of Dbl alone which, although expressed, did not complement the cdc24-m1 mating defect. The Vav protein, did not display any effect on the mating defect. This lack of effect may be due to either insufficient expression of the Vav protein or to the fact that Vav function requires a phosphorylation of the Lck kinase which must be co-expressed with the Vav protein before an effect can be observed.

E) Assays to Determine FAR1 Interaction With Cdc24p and Gβ

Studies have shown that FAR1 may play an important role both for pheromone mediated growth arrest and growth orientation during mating (Valtz, N. , Peter, M. & Herskowitz, I J. Cell Biol. 131, 863-73 (1995); Chang, F. & Herskowitz, I. Cell. 63, 999-1011 (1990); Peter, M., Gartner, A. , Horecka, J. , Ammerer, G. & Herskowitz, I. Cell 73, 747-60 (1993)). The orientation function, which is specifically disrupted in a far1-H7 mutant is required for the Cdc24 G,β interaction suggesting that Far1 might interact with Cdc24. Two-hybrid analyses show that indeed Far1 interacts with Cdc24.

White the Cdc24 Gβ interaction requires the presence of FAR1, the Far1 Cdc24 interaction is independent of Gβ suggesting that Far1 might bind Cdc24 directly whereas Cdc24 Gβ are part of a complex which include Far1. Far1 also interacts by twp-hybrid assays with Gβ, consistent with the notion that Cdc24, Far1, and Gβ form a complex. In a diploid two-hybrid strain, in which a number of pheromone response genes are not expressed, we are unable to detect the Cdc24 Gβ interaction. However, overexpression of tarl results in an interation and further overexpression of Gγ results in a maximal Interaction, indicating that a complex comprised of Cdc24, Gβγ, and Far1 forms even in diploid cells.

Although cdc24-m and far1-s mutants result in similar defects in growth orientation during mating, we set out to determine if these genes function in the same orientation process. Generation of a cdc24-m1 mutation in a Δfar1 strain did not result in a substantial decrease in mating efficiency, suggesting these two genes function in the same process. In contrast, results from double mutants of cdc24-m1 with Δspa2, Δste20, or ΔbemI suggest that these three genes do not function in the same orientation process as Cdc24 and Far1. Cdc24 and Far1 were epitope tagged in order to determine whether these proteins interact in yeast cells. The chromosomal copy of Cdc24 was replaced with a 3xmyc tagged Cdc24 and the chromosomal copy of Far1 was replaced with Far1 protein A fusion. Both of these fusion proteins are fully functional. Isolation of Far1-protein A from yeast extracts using IgG-Sepharose co-precipitated 3xmyc-Cdc24. In contrast, the 3xmyc-Cdc24-m1 mutant was defective in binding Far1 in similar immunoprecipitation assays. These results indicate that Cdc24 and Far1 bind one-another and this interaction may be essential for growth orientation during mating.

Far1 Binds Cdc24 and Gβ

The binding relationships between Cdc24, Far1, and Gβ were examined in vitro using proteins purified from bacteria and yeast. Gβγ was purified from yeast cells using a chromosomal copy of the gene which has HA epitope (Tyr-Pro-Tyr-Asp-. Val-Pro-Asp-Tyr-Ala) fused to the amino-terminus and protein A fused to the carboxyl-terminus. A tobacco etch Tirus (TEV) protease cleavage site (recognition site Glu-Asn-Leu-Tyr-Phe-Gl-Gly with cleavage occurring between Gin and Gly) was placed between Gβ and the protein A domain so that material isolated from yeast using IgG-Sepharose can be specifically eluted with commercially available recombinant TFV protease. Maltose. binding protein (MBP) Far1 fusions have been expressed and purified from E. coli. Similarly, a glutathione-S-transferase (GST) Cdc24 fusion (residues 1-472) has been expressed and pfied from E. coli. MBP-Far1 binds GST-Cdc24 specifically. The removal of the 75 carboxyl-terminal residues of Far1 (H7) prevents Cdc24 binding. Furthermore GST alone is unable to bind MBP-Far1.

These results show that Cdc24 can directly bind Far1 in the absence of any other yeast proteins. Far1 fragments containing either the amino-terminal Lim domain (a domain implicated in protein-protein interactions) or the carboxyl-terminus were tested for their ability to bind GST-Cdc24. Both fragments showed very little binding to GST-Cdc24 indicating that although the Far1 carboxyl-terminus is necessary, it is not sufficient for 0dc24 binding. Using MBP-Far1 we have been able to observe binding to Gβ purified from yeast. Binding of Gβ is reduced using amino-terminal or carboxy-terminal MBP-Far1 fragments, yet Gβ binds Far1H7 as well as Far1.

In one preferred aspect of the present invention the assay also includes the presence of Far1.

TABLE 1
cdc24-ml is defective in cell mating

| Strain | Tester | % Mating efficiency |
|---|---|---|
| CDC24 MATα | MATa WT | 100 (21) |
| cdc24-ml MATα | MATa WT | 0.5 (0.2) |
| CDC24 MATa | MATα WT | 100 (20) |
| cdc24-ml MATa | MATα WT | 3.8 (1.6) |
| CDC24 MATa | MATα Δfus1 Δfus2 | 100(17) |
| cdc24-ml MATa | MATα Δfus1 Δfus2 | ≦0.02 |
| CDC24 MATa | CDC24 MATα | 100(18) |
| cdc24-ml MATa | cdc24-ml MATα | ≦0.0006 |

Mating efficiencies are the number of diploid cells divided by the total cells with CDC24 WT set to 100%. The values are means of 4 determinations with standard deviation ( ). Absolute mating efficiency was 14-15% with MATa and MATαtesters, 1.8% with Δfus1 Δfus2 tester, and 3.4% with CDC24 tester,

Some of the results are also shown in the accompanying Figures. These Figures are now discussed in more detail.

FIG. 1

cdc24-m1 phenotypes. a, Actin cytoskeleton of cdc24-m1 cells shows polarised distribution. Bar equals 5 mm. b, Pheromone-induced growth arrest is similar in cdc24-m1 wit WT cells. Sterile filter disks spotted with α-factor (1, 0.5, 0.2, 0.1, 0.05, and 0.012 mg) were placed onto cells in agarose. c, MAP-kinase pathway signaling is uninfected in cdc24-m1. LacZ activities are the average of 2 experiments (2-3 determinations per experiment) with standard deviation. WT maximum (29.6 Miller Units) was set to 100%.

FIG. 2

cdc24-m1 cells are unable to orient in a pheromone gradient. a, Excess pheromone has a negligible effect on cdc24-m1 mating. U47a cells were mated with a WT tester and mating efficiency for CDC24 (2.8%) was set to 100%. Values are means (n=2). b, cdc24-m1 cells are unable to orient in a pheromone gradient. A trace of cell shapes after 6-7 hr in a pheromone gradient is shown with arrowheads indicating orientation. Quantitation of cell projection angle relative to the inicropipet (needle) from 4-7 separate experiments (n=112 CDC24 and 167 cdc24-m1 cells)-The average cosine of the angle of cell projection relative to the micropipet was 0.52 for CDC24 and-0.02 for cdc24-m1 cells (a cosine of 1 represents perfect orientation and 0, random orientation). c, cdc24-m1 cells position their shmoos adjacent their bud scar. The position of the bud scar on zygotes was determined for aproximately 120 cells.

FIG. 3

cdc24-m mutants are defective in mating and Ste4p (Gβ) binding. a, Location of cac24p mating mutations. Mat patches show diploids from mating with MATa WT tester. Ste4 2-H patch growth on-leu-trp-his indicates an interaction of Cdc24p (1-288 aa) with Ste4p. Similar results were obtained using a LacZ reporter in strain Y187 (relative Miller Units 100 for Cdc24/Ste4 and 3 for Cdc24-m1/Ste4). b, Two hybrid interactions of Cdc24p. For interactions with Ste4p, a fragment of Cdc24p (1-288 aa) was used, however, full length Cdc24p also interacts with Ste4p. c, Region of Cdc24p necessary for Ste4p interaction. Numbers refer to Cdc24p aa fused to DBD. d, Cdc24p bins to Ste4p in the absence of other yeast proteins. Mixed bacterial cell extracts (1 eq) containing either His₆Ste4p and GST or GSTCdc24p (1-472 aa), and GSH-agarose eluates (800 eq) were separated by SDS-PAGE, immuno-blotted and probed with anti-sera to His₆Ste4p, Anti-GST sera showed similar amounts of GST and GSTCdc24p in eluates, Due to proteolysis, His₆Ste4p migrates as a doublet.

FIG. 4

Model for signal transduction pathway required for cell orientation. For clarity we have omitted components of MAP-kinase cascade. The role of Cdc42p (a Rho-family GTPase) in cell orientation is speculative. Pheromone binds the pheromone receptor (Ste2p or Ste3p) resulting in the dissociation of Gα (Gpa1p) from Gβγ (Ste4p/Ste18p). Direct binding of Cdc24p to Gβγ0 (in the vicinity of the receptor) activates or recruits Cdc42p which is necessary for oriented growth towards a mating partner.

SEQUENCE ANALYSIS

The DH and PH sequences were analysed by a Blast homology search. In addition, an. analysis of the amino acid identity over the entire protein to S. cerevisia Cdc24p was conducted. DH refers to the Dbl homology region (GEF region)—see Hart et al 1991 Nature 354 311-314; Miyamoto et al 1991 Biochem Biophys Res Commun M181 604-610; Ron et al 1991 New Biol 3 372-379. PH refers to the Pleckstrin homology region—see Musacchio et al Trends Biochem Sci 18 343-348

The results are as follows:

A. Blast Homology Search Using Cde24 DH and PH Region TBLASTN 1.4.9 MP

Query=yeast Cdc24p DH PH (392 aa):

KIIKEFVATERKYVHDLEILDKYRQQLLDSNLITSEELYMLFPNLGDAIDFQRRFLISLEI
NALVEPSKQRIGALFMHSKHFFKLYEPWSIGQNAAIEFLSSTLHKMRVDESQRFIINNKL
ELQSFLYKPVQRLCRYPLLVKELLAESSDDNNTKELEAALDISKNIARSINENQRRTEN
HQVVKKLYGRVVNWKGYRISKFGELLYFDKVFISTTNSSSEPEREFEVYLFEKIIILFSE
VVTKKSASSLILKKKSSTSASISASNITDNNGSPHHSYHKRHSNSSSSNNIHLSSSSAAAII
HSSTNSSDNNSNNSSSSSLFKLSANEPKLDLRGRIMIMNLNQIIPQNNRSLNITWESIKEQ
GNFLLKFKNEETRDNWSSCLQQLIHDLKN

Database: Non-redundant Genbank+EMBL+DDBJ+PDB sequences 349,525 sequences; 540,957,745 total letters

Reference: Altsehul, Stephen F. , Warren Gish, Webb Miller, Engene W. Myers, and David J. Lipman (1990). Basic local alignment search tool. J. Mol. Biol. 215:403-410.

| | | | | Smallest | Smallest |
|---|---|---|---|---|---|
| | | | | Sum Prob- | Sum Prob- |
| | | Reading | High | ability | ability |
| | | Frame | Score | P(N) | N |
| gb\|U12538\|SPU12538 | Schizosaccharomyces | +3 | 171 | 1.0e−51 | 6 |
| | pombe scd1 | | | | |
| emb\|X57298\|MMMCF2PO | M.musculus Mcf2 | +1 | 128 | 8.3e−10 | 3 |
| | proto-oncogene | | | | |
| | (Mcf2 is Dbl) | | | | |
| gb\|U16296\|HSU16296 | Human T-lymphoma | +3 | 88 | 2.3e−09 | 3 |
| | invasion and metastasis | | | | |
| | inducing TIAM1 | | | | |
| gb\|U05245\|MMU05245 | Mus musculus BALB/c | +3 | 88 | 5.5e−09 | 3 |
| | invasion inducing | | | | |
| | protein (Tiam-1) | | | | |
| gb\|J03639\|HUMDBLTP | Human DBL oncogene | +2 | 121 | 2.1e−07 | 3 |
| | encoding a transforming | | | | |
| | protein | | | | |
| gb\|S76992\|S76992 | VAV2═VAV oncogene | +3 | 125 | 2.6e−07 | 2 |
| | homolog human | | | | |
| dbj\|D86547\|D86547 | Fruitfly still life type 1 | +2 | 76 | 5.4e−07 | 5 |
| gb\|U37017\|MMU37017 | Mus musculus Vav2 | +1 | 126 | 6.4e−07 | 2 |
| | oncogene | | | | |
| dbj\|D86546\|D86546 | Fruitfly still life type 2 | +1 | 76 | 1.0e−06 | 5 |
| gb\|U39476\|RNU39476 | Rattus norvegicus p95 | +3 | 116 | 6.3e−06 | 1 |
| | Vav proto-oncogene | | | | |
| gb\|S76838\|S76838 | Dbs (Dbl guanine | +3 | 112 | 4.4e−05 | 2 |
| | nucleotide exchange | | | | |
| | factor homolog) murine | | | | |
| dbj\|AB002360\|AB002360 | Human KIAA0362 | +2 | 113 | 4.5e−05 | 2 |
| emb\|Z35654\|RNOSTOG | R.norvegicus Ost | +1 | 112 | 4.9e−05 | 2 |
| | oncogene | | | | |
| emb\|X83931\|HSVAVONCO | H.sapiens VAV | +1 | 109 | 5.5e−05 | 1 |
| | oncogene | | | | |
| gb\|AF003147\|CELC11D9 | Caenorhabditis elegans | +3 | 81 | 0.0070 | 3 |
| | C11D9 | | | | |
| gb\|U96634\|MMU96634 | Mus musculus p85SPR | +2 | 62 | 0.016 | 3 |
| emb\|Y10159\|DDY10159 | D.discoideum racGAP | +1 | 71 | 0.025 | 3 |
| gb\|U58203\|MMU58203 | Mus musculus Lsc | +2 | 75 | 0.044 | 2 |
| | oncogene | | | | |
| emb\|Y09160\|HSSUB15 | H.sapiens Sub1.5 | +1 | 80 | 0.063 | 2 |
| gb\|AF003740\|CELC41D11 | Caenorhabditis elegans | +2 | 81 | 0.064 | 4 |
| | C41D11 | | | | |
| gb\|U02081\|HSU02081 | Human guanine | +1 | 77 | 0.12 | 2 |
| | nuoleotide regulatory | | | | |
| | protein (NET1) | | | | |
| gb\|U00055\|CELR02F2 | Caenorhabditis elegans | +1 | 85 | 0.13 | 1 |
| | R02F2 | | | | |
| gb\|U64105\|HSU64105 | Human guanine | +1 | 77 | 0.14 | 1 |
| | nucleotide exchange | | | | |
| | factor p115-RhoGEF | | | | |
| gb\|U42390\|HSU42390 | Homo sapiens Trio | +1 | 74 | 0.33 | 3 |
| gb\|M24603\|HUMBCRD | Human bcr protein | +1 | 58 | 0.91 | 3 |
| | amino end | | | | |
| emb\|X02596\|HSBCRR | Human bcr (breakpoint | +3 | 58 | 0.996 | 3 |
| | cluster region) in | | | | |
| | Philadelphia | | | | |
| | chromosome | | | | |
| gb\|U11690\|HSU11690 | Human faciogenital | +2 | 73 | 0.999 | 1 |
| | dysplasia (FGD1) | | | | |
| gb\|U22325\|MMU22325 | Mus musculus | +3 | 73 | 0.9997 | 2 |
| | faciogenital dysplasia | | | | |
| | (Fgd1) | | | | |
| gb\|M15025\|HUMBCRABL | Human BCR/ABL | +3 | 58 | 0.99995 | 5 |
| | product of the | | | | |
| | translocation of | | | | |
| | t(222q11; 9q34) | | | | |

| | | protein | % identity |
|---|---|---|---|
| Organism | gene | size (aa) | (aa) |
| Schizosaccharomyces pombe | Scd1 | 834 | 21.9 |
| Mouse | Fgd1 | 960 | 16.7 |
| Human | Fgd1 | 961 | 16.5 |
| Mouse | Vav2 | 868 | 16.5 |
| Mouse | Ect2 | 768 | 16.2 |
| Human | Vav2 | 878 | 15.8 |
| Worm | Q18479 | 860 | 15.4 |
| Mouse | Vav | 844 | 14.6 |
| Rat | Vav | 843 | 14.5 |
| Human | Vav | 846 | 14.4 |
| Mouse | Dbs | 1150 | 14.3 |
| Human | Tim | 519 | 14.0 |
| Human | proto-Dbl | 925 | 13.4 |
| Human | p115RhoGEF | 912 | 13.4 |
| Mouse | Lfc | 572 | 13.4 |
| Rat | Ost | 872 | 12.9 |
| Worm | Q22354 | 862 | 12.9 |
| Mouse | Lsc | 919 | 12.5 |
| Human | Lbc | 424 | 12.4 |
| Human | Net1 | 460 | 12.3 |
| Human | BCR | 1271 | 11.9 |
| Mouse | Tiam1 | 1591 | 11.2 |
| Human | Tiam1 | 1591 | 10.9 |
| Mouse | proto-Dbl | 320(partial) | 9.7 |
| Drosophila | Still Life 1 | 2064 | 9.0 |
| Drosophila | Still Life 2 | 2044 | 8.4 |

B. Amino acid identity over entire protein to S. cerevisiac Cdc24p

Protein name key;

Scd1: Schizosaccharomyce pombe Cdc24¹⁰¹.

Fgd1 Faciogeniate Dysplasia Protein. FGD also known as Aarskog-Scott syndrome, is an X-linked developmental disorder¹⁰².

Vav/Vav2 A oncogene derived from hematopoietic cells¹⁰³.

Q18479 (similar to Vav)

Q22354 (similar to Vav)

Ect2 Oncogene expressed in epithelial cells and possessing transfonning potential¹⁰⁴.

Tim Mammary epithelial oncogene¹⁰⁵.

Dbl/Dbs Diffuse b-cell lymphoma (dbl) oncogene^{106,107}.

p115RhoGEF Regulates cell proliferation, induces the transformation of cells¹⁰⁸.

Lfc Heratopoictic oncogene¹⁰⁹.

Ost Osteosarcoma derived proto-oncogene. Truncation is oncogenic and highly tumonigenic in mice¹¹⁰.

Lsc Oncoprotein¹¹¹.

Lbc ocogene involved in chronic myeloid leukemias¹¹².

Net1 Neuroepithelioma transforming oncogene¹¹³.

BCR bcr (breakpoint cluster region), an oncogene which is the tanslocation breakpoint in chronic mycloid leukemias(CML)^{114,115}.

Tiam1 Human invasion-and metastasis-inducing tiam1 gene and is expressed in tumor-cell lines of different tissue origin¹¹⁶.

Still Life 1/2 A synaptic terail protein¹¹⁷.

### DISCUSSION

CDC42 and its GDP/IGTP exchange factor (GEF) CDC24 are required for vegetative growths^{8,9} and cell mating^{6,7,10}. The precise function of these proteins in cell mating has been difficult to study because icy are essential for viability. In accordance with the present invention, we reasoned that if CDC24 has a specific function in the mating pathway, cdc24 alleles should exist which affect cell mating but not vegetative growth. To identfy such alleles, a collection of CDC24 random mutants was screened and thre recessive mating mutants, cdc24-m1-3 were isolated (FIG. 3A). This screen required isolated cdc24 mutants to be able to support vegetative growth. Further characterization of cdc24-m cells revealed normal growth between 18°and 37° and cell morphology, bud site selection, and acti disibution were similar to WT cells (see below and FIG. 1A). The specificity of the cdc24-m phenotype is in contrast to that of all other described cdc24 mutants which have strong defects in vegetative growth⁸⁻¹⁰.

To elucidate the role of CDC24 in mating, we examined cdc24-m1 cells for defects in the mating pathway. The mating efficiency of cdc24-m1 cells with a WT partner was reduced approximately 100-fold compared to WY (Table 1), and this effect was essentially independent of mating type. When cdc24-m1 or an enfeebled mater defective in cell fusion were used as mating partners, significantly stronger defects were observed. Such bilateral mating defects suggest impairment in a process such as shmoo (mating projection) formation, orientation, or fusion in which a WT mating partner can partially compensate for the mutant stain.

Pheromone activation results in a number of responses including cell cycle arrest, MAP. -kinase cascade mediated induction of mating. specific genes, and changes in cell morphology ^{4,5}. Pheromone-induced growth arrest deter by halo-assays showed both cdc24-rnl and WT cells responded similarly (FIG. 1B). Furthermore, overexpression of the β-subunit of the yeast hetero-timeric G-protein, Ste4p, from an inducible promoter arrested growth of both cdc24-m1 and WT cells (data not shown). Microscopic examination revealed identical umbers of WT and cdc24-mi cells (78%, n=1600) formed shmoos afer 4 hr exposure to 10 mM pheromone. The acting distribution of cdc24-m1 budding and shmooing cells was also similar to that of WT cells (FIG. 1A), demonstrating that the mating defect was not due to an inability to polarise the acting cytoskeleton. The level of pheromone induced FUS-lacZ expression, a reporter used to measure induction of mating specific genes¹¹, was similar in cdc24-m1 and WT cells FIG. 1C). However, examination of mating mixtures of cdc24-m1 and WT tester cells showed a greater th ten-fold decrease in the number of zygotes, indicating that the cdc24-m1 defect occurs prior to cell fusion. Thus cdc24-m cells appear normal for cell cycle arrest, shmoo formation, actin cytoskeleton polarisation, and MAP-kinase signaling, yet are defective at a step prior to cell fusion.

During mating, polnised growth towards a mating parther requires a pheromone gradient¹² and saturation with pheromone during mating results in random orientation of growth and mating partner selection, and hence a decrease in mating efficiency^{13,14}. WT cells showed a 16-fold decrease in mating efficiency in the presence of satating pheromone (20 M), whereas only 10% reduction was observed with cdc24-m1 cells (FIG. 2A), suggesting that this mutant is unable to orientate towards a pheromone gradient during mating. Similar results were observed with cdc24-m2 and cdc24-m3 cells. To test directly whether cdc24-m1 cells are defective in mating projection orientation their response to an artificial pheromone gradient created by a micropipet was examined. While CDC24 cells oriented growth towards the pheromone source (greater than 70% of cells oriented within 60° angle of micropipet), cdc24-m1 cells did not show a preferred orientation (FIG. 2B). No difference in the sensitivity of WT or mutant cells to pheromone was observed

Although cdc24-m1 cells oriented randomly in a pheromone grient, the choice of shmoo site could be dictated by an internal cue, such as the previous bud site. To examine this possibility, the location of the bud scar (in cells with a single bud scar) relative to the neck of the zygote was determined. While WT cells showed a random position of their bud scar on the zygotes, 86% of cdc24-m1 zygotes had formed a shmoo adjacent to their previous bud site (FIG. 2C). Together these results establish a specific role for Cdc24p in orientation towards a mating partner.

Sequencing of cdc24-m alleles revealed mutations that changed one of two adjacent amino acid residues (FIG. 3A). cdc24-m1 and cdc24-m3 both have a single amino acid change from Ser 189 to either a Phe or Pro. cdc24-m2 had two amino acid substitutions and subcloning demonstrated that the mutation responsible for the mating defect is Asp to Gly at residue 190. The grouping of these mutations suggests that this region of Cdc24p is important for an interaction required for oriented grow.

Previous two-hybrid studies have suggested that the amino-terminus of Cdc24p might interact with Ste4p⁷, however, the in vivo significance of this association was unclear. We determined whether Cdc24p mating mutants could interact with Ste4p (FIG. 3A). In contrest to the wild-type Cdc24p, the mutants did not show a detectable interaction with Ste4p. In agreement with the clustering of the cde24-m mutations, amino acid residues 170 to 245 of Cdc24p were sufficient for the Ste4p interaction (FIG. 3C), while an amino-terminal gent consisting of the first 160 a o acid residues, although expressed, failed to intect. Coonsistent with a functional significance of the Cdc24p Ste4p interaction, we have isolated mutants in STE4, (exemplified by SEQ ID No. 9 and SEQ ID No. 10), using a two-hybrid screen, which are unable to interact with Cdc24p and are phenotypically similar to cdc24-m raus.

To assess the specificity of the defect in the interaction between Ste4p and Cdc24-m1p, interactions with Cdc42p and Bem1p, two proteins known to bind to Cdc24p^{15,16} were investigated Bem1p is an SH3 domain protein involved in bud formation and mating¹⁷. Cdc24-m1p was able to interact with both Cdc42p and Bem1p (FIG. 3B) consistent with the absence of an effect of cdc24-m1 on vegetative growth.

While the cdc24-m1 phenotype along with the two-hybrid results: indicates that the interaction between Cdc24p and Gβ is central to cell orientation, these results do not address whether this interaction is direct or indirect. Gβ typically functions as a complex with e third subunit of a hetero-trimeric G-protein, Gγ. We therefore determined whether the yeast Gγ, Ste18p, was required for the Cdc24p Ste4p interaction. Deletion of STE18 abolished the Cdc24p Ste4p two-hybrid interaction (data not shown), suggesting at Cdc24p interacts with the Gβγ-complex. To determine if Cdc24p could directly bind Ste4p, these proteins were expressed in bacteria Hexahistidine-tagged Ste4p specifically bound to GSTCdc24p (FIG. 3D). These results demonstrate fhat Cdc24p an directly bind Gβ in the absence of any other yeast proteins. We attribute the requirement for Gγ in the two-hybrid assays to its stabisation of Gβ¹⁸.

Pheromone receptor activation results in dissociation of Gβγ from Gα at the receptor. Our results indicate that the orientation defect in cdc24-m cells is due to a specific defect in the Cdc24p Gβγ interaction. This suggests a model in which direct binding of Cdc24p to Gβγ results in recruitment (to the vicinity of the receptor) or activation of Cdc42p and that this local concentration of activated Cdc42p is responsible for oriented growth towards a pheromone gradient (FIG. 4). In the absence of this recruitment or activation a site adjacent to the previous bud site appears to function as a default site for shmoo formation. Our results together with previous studies implicating Cdc24p in bud site selection⁸, suggest that Cdc24p acts as a crucial component required both for bud and shmoo site selection, perhaps functioning as a kind of molecular selector switch between internal signals for bud site selection and external signals for shmoo site selection. It is likely that local activation of Cdc24p recruits and activates the Rho GTPase Cdc42p, which could then interact with downstream targets required for orientation of the cytoskeleton. Cdc42p interactions with the protein kinase Ste20p^{19,20} are not necessary for cell orientation²⁰,suggesting that novel targets of Cdc42p are required for oriented growth towards a mating parter

Cdc24p belongs to a diverse family of GEFs which include many rninalian proto-oncogenes². This group of proteins shares a conserved region consisting of a Dbl-domain (named after the human proto-oncogene Dbl) followed by a plecktstrin-homology domain (PH). Sequeace comparison revealed similarity between a small stretch of amino acids flanking the cdc24 mating mutations and Dbl (FIG. 3A). Our results indicate that an association between Cdc24p and Gβγ links pheromone receptor activation to shmao orientation. We propose that other GEFs, such as the proto-oncogene Dbl, provide a similar connection between G-protein coupled receptor activation and polarised cell growl

Hence, in accordance with the present invention there are provided the following uses and utilites of Cdc24p/Ste4 interaction and cdc24-m mutants

1) Peptide inhibitors which block GEF/Gβ interaction. Peptides and peptidyl derivatives based regions encompassing niutants will be used to block and/or antagolnise GEF (such as the proto-oncogenes Dbl or Vav) Gβ interaction. Derivatives of these peptides (including peptide mimics) which bind with higher affinity will also be used. The perturbation of these interactions will be of therapeutic value for example in treatment of cancers.

2) Simple yeast based assays systems (utilising matin function and interaction reporters) will be extremely useful for high through-put screening to identify molecules perturbing this GEF/Gβ interaction. In particular, the qualitative effect on mating observed with the proto-oncogene, proto-Dbl, even at low levels of expression, indicates that this type of assay is amenable to large scale screening for the effect of agents, such as proto-oncogenes, on induced defects in yeast and other host cells.

3) Similar Cdc24p/Gβ interactions will be ideal targets for anti-fungal drugs directed at the pathogenic yeast Candida.

### SUMMARY

1) We have identified an impotat interaction between two general cellular components, Cdc24p and Gβ f which provides a connection between G protein coupled receptor activation and polarised cell growth. This work has been exemplified by work done with yeast genes/proteins, however, both celluar components involved have homologues in humans.

2) We show the physiological consequence of this interaction and from these data extrapolate to the general role of the interaction in human cells.

3) In addition, we have identified sequences required for this interaction Specifically, we have identified a short stretch of one protein (Cdc24p) encompassing 75 aa sufficient for this interaction and three amino acid changes (within this stretch) which block the interaction and have physiological consequences These amino acid changes fall within a 19 amino acid piece with similarity to the human proto-ongene Dbl. Indeed removal of this region from proto-Dbl (when the amino terminus is removed) results in oncogenicity in tissue culture cells.

4) We have also identified specific mutants in the Bβ-subunit of the heterodimieric G protein (Ste4p) which appear to block its interaction with Cdc24p. We believe that several of these mutations will fall in conserved retions of Gβ. Thus, it is possible to devise assays based on this mutation to screen for agents capable of modifying the non-interactive behaviour of the mutant G protein β subunit with Cdc24p. In addition, the assay could be used to study Cdc24p honmologues or even Cdc24p derivatives or homologues to see if those derivatives or homologues affect the non-interactive behaviour of the mutant G protein. ,

5) There is a wealth of information on human Gβ's, human GEF's GDP/GTP Exchange Factors), such as Cdc24p homologues and the rho family of GTP-binding-proteins (such as rho like Cdc42p) which the GEFs work on. Most human GEF's are oncogenes such as Dbl, Vav, and Edt and are involved in some way in growth control. Furthemore Gβ's are involved in linking signals from receptors to ultracelullar responses. The present invention has shown that that a GEF from yeast, Cdc24p, can directly bind Gβ in the absence of any other yeast proteins. Although unproven, it is likely that inflations between human GEF's and Gβ's are also crucial im growth control and chemotaxis.

6) We propose the interaction we have identified will have broad cellular ramifications and manipulation of these interactions (such as peptidic inhibitors anld peptides mimicking activated species) will be of therapeutic value.

7) In addition, simple yeast based assays systems could be extremely useful for high through-put screening to identify molecules perturbing this interaction. In particular, a qualitative assay using a yeast mutant with a mating defect could prove useful in the design of agents, such as anti-cancer agents, that can affect the function of orcogenes such as proto-Dbl, in terms of its ability to complement a yeast mutant mating defect and/or its function in mammalian tissue culture cells.

8) We also believe similar interactions will be ideal targets for anti-fungal drugs directed at invasive and pathogenic yeasts such as Candida albicans and Cryptococcus neoformans.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific emnbodiments. Indeed, various modifications of the described modes for carrying out the invention which are obvious to those skilled in molecular biology or related fields are intended to be within the scope of the following claims.

What is claimed is:

1. An isolated polynucleotide encoding a protein having a capability of substantially affecting the interaction of Gβ with Cdc24p or a homologue thereof that is capable of associating therewith.

2. An isolated polynucleotide according to claim 1, said polynucleotide comprising nucleotides in the sequence shown as SEQ ID NO: 1.

3. The polynucleotide of claim 1, wherein said polynucleotide comprises a sequence selected from the group consisting of SEQ ID NO: 3, SEQ ID NO: 5 and SEQ ID NO: 7.

4. The polynucleotide of claim 1, wherein said protein encoded by said polynucleotide has a sequence selected from the group consisting of SEQ ID NO: 4, SEQ ID NO: 6 and SEQ ID NO: 8.

5. An isolated polypeptide that can affect the interaction of a Gβ and a Cdc24p or which is capable of associating with the Cdc24p or a homologue thereof.

6. An isolated polypeptide according to claim 5, said polypeptide comprising amino acids having the sequence of SEQ ID NO: 2.

7. An isolated polypeptide according to claim 5, said polypeptide comprising amino acids having a sequence selected from the group consisting of SEQ ID NO: 4, SEQ ID NO: 6 and SEQ ID NO: 8.

8. A kit for identifying an agent that affects growth behavior of cells, comprising:
a polynucleotide according to claim 2; and
a Gβ that is capable of associating with a Cdc24p or a homologue thereof.

9. The kit of claim 8, wherein said polynucleotide has a sequence other than that of SEQ ID NO: 1.

10. The kit of claim 9, wherein the polynucleotide comprises a sequence selected from the group consisting of SEQ ID NO: 3, SEQ ID NO: 5 and SEQ ID NO: 7.

11. A kit for identifying an agent that affects growth behavior of cells, comprising:
a polypeptide according to claim 5; and
a Gβ that can associate with a Cdc24p or a homologue thereof.

12. A kit according to claim 11, wherein the polypeptide has a sequence other than that of SEQ ID NO: 2.

13. A method for identifying an agent that affects growth behavior of cells, comprising the steps of:
contacting the agent with a polynucleotide according to claim 1 in the presence of a Gβ that is capable of associating with Cdc24p or a homologue thereof;
determining whether the agent affects the interaction of the polynucleotide with the Gβ ; and
identifying the agent as one which affects the growth behavior of cells if the agent affects the interaction of the polynucleotide and the Gβ.

14. A method according to claim 13, wherein the polynucleotide has a sequence other than that of SEQ ID NO: 1.

15. An method for identifying an agent that affects growth behavior of cells, comprising the steps of:
contacting an agent with a polypeptide according to claim 5;
determining whether the agent affects the interaction of the protein and Gβ or a Rho-family GTPase; and
identifying the agent as one which affects the growth behavior of cells if the agent affects the interaction of said polypeptide and the Gβ or Rho-family GTPase.

16. A method according to claim 15, wherein the polypeptide has a sequence other than that of SEQ ID NO: 2.

17. A method according to claim 16, wherein the polypeptide has a sequence selected from the group consisting of SEQ ID NO: 4, SEQ ID NO: 6 and SEQ ID NO: 8.

18. An isolated polypeptide having a sequence selected from the group consisting of: SEQ ID NO: 15, SEQ ID NO: 16, SEQ ID NO: 17, SEQ ID NO: 18 and SEQ ID NO: 19.

19. An assay method for identifying an agent as capable of affecting the of Cdc24p or a homolog thereof with a Gβ or an associated Rho-family comprising the steps of:
contacting said agent with a polypeptide sequence comprising a sequence of one of SEQ ID NOs:15-19 or a derivative, fragment, variant or olog thereof; and
determining whether said agent is capable of affecting said interaction.


SEQUENCE LISTING (20 sequences)

SEQ ID NO: 1 - DNA, 228 bp, S. cerevisiae
cccctctgtatacttttcaactctgtgaagccgcaatttaaattaccggtaatagcatctgacgatttgaaagtctgtaaaaaatccatttatgactttatattgggctgcaagaaacactttgcatttaacgatgaggagcttttcactatatccgacgtttttgccaactcgacgtcccagctggtcaaagtgctagaagtagtagaaacgctaatgaattccagc

SEQ ID NO: 2 - protein, 76 aa, S. cerevisiae
PLCILFNSVKPQFKLPVIASDDLKVCKKSIYDFILGCKKHFAFNDEELFTISDVFANSTSQLVKVLEVVETLMNSS

SEQ ID NO: 3 - DNA, 228 bp, Artificial Sequence
  cdc24m1 mutant.
cccctctgtatacttttcaactctgtgaagccgcaatttaaattaccggtaatagcatttgacgatttgaaagtctgtaaaaaatccatttatgactttatattgggctgcaagaaacactttgcatttaacgatgaggagcttttcactatatccgacgtttttgccaactcgacgtcccagctggtcaaagtgctagaagtagtagaaacgctaatgaattccagc

SEQ ID NO: 4 - protein, 76 aa, Artificial Sequence
  cdc24m1 mutant.
PLCILFNSVKPQFKLPVIAFDDLKVCKKSIYDFILGCKKHFAFNDEELFTISDVFANSTSQLVKVLEVVETLMNSS

SEQ ID NO: 5 - DNA, 228 bp, Artificial Sequence
  cdc24m2 mutant.
cccctctgtatacttttcaactctgtgaagccgcaatttaaattaccggtaatagcatctggcgatttgaaagtctgtaaaaaatccatttatgactttatattgggctgcaagaaacactttgcatttaacgatgaggagcttttcactatatccgacgtttttgccaactcgacgtcccagctggtcaaagtgctagaagtagtagaaacgctaatgaattccagc

SEQ ID NO: 6 - protein, 76 aa, Artificial Sequence
  cdc24m2 mutant.
PLCILFNSVKPQFKLPVIASGDLKVCKKSIYDFILGCKKHFAFNDEELFTISDVFANSTSQLVKVLEVVETLMNSS

SEQ ID NO: 7 - DNA, 228 bp, Artificial Sequence
  cdc24m3 mutant.
cccctctgtatacttttcaactctgtgaagccgcaatttaaattaccggtaatagcacctgacgatttgaaagtctgtaaaaaatccatttatgactttatattgggctgcaagaaacactttgcatttaacgatgaggagcttttcactatatccgacgtttttgccaactcgacgtcccagctggtcaaagtgctagaagtagtagaaacgctaatgaattccagc

SEQ ID NO: 8 - protein, 76 aa, Artificial Sequence
  cdc24m3 mutant.
PLCILFNSVKPQFKLPVIAPDDLKVCKKSIYDFILGCKKHFAFNDEELFTISDVFANSTSQLVKVLEVVETLMNSS

SEQ ID NO: 9 - protein, 392 aa, Artificial Sequence
  STE4 mutant.
KIIKEFVATERKYVHDLEILDKYRQQLLDSNLITSEELYMLFPNLGDAIDFQRRFLISLEINALVEPSKQRIGALFMHSKHFFKLYEPWSIGQNAAIEFLSSTLHKMRVDESQRFIINNKLELQSFLYKPVQRLCRYPLLVKELLAESSDDNNTKELEAALDISKNIARSINENQRRTENHQVVKKLYGRVVNWKGYRISKFGELLYFDKVFISTTNSSSEPEREFEVYLFEKIIILFSEVVTKKSASSLILKKKSSTSASISASNITDNNGSPHHSYHKRHSNSSSSNNIHLSSSSAAAIIHSSTNSSDNNSNNSSSSSLFKLSANEPKLDLRGRIMIMNLNQIIPQNNRSLNITWESIKEQGNFLLKFKNEETRDNWSSCLQQLIHDLKN

SEQ ID NO: 10 - DNA, 1269 bp, Artificial Sequence
  STE4 mutant.
atggcacatcagatggactcgataacgtattctaataatgtcacccaacagtatatacaaccacaaagtctacaggatatctctgcagtggaggaagaaattcaaaataaaatagaggccgccagacaagagagtaaacagcttcatgctcaaataaataaagcaaaacacaagatacaagatgcaagcttattccagatggccaacaaagttacttcgttgaccaaaaataagatcaacttaaagccaaatatcgtgttgaaaggccataataataaaatctcagattttcggtggagtcgagattcaaaacgtattttgagtgcaagtcaagatggctttatgcttatatgggacagtgcttcaggtttaaaacagaacgctattccattagattctcaatgggttctttcctgcgctatttcgccatcgagtactttggtagcaagcgcaggattaaacaataactgtaccatttatagagtttcgaaagaaaacagagtagcgcaaaacgttgcgtcaattttcaaaggacatacttgctatatttctgacattgaatttacagataacgcacatatattgacagcaagtggggatatgacatgtgccttgtgggatataccgaaagcaaagagggtgagagaatattctgaccatttaggtgatgttttggcattagctattcctgaagagccaaacttagaaaattcttcgaacacattcgctagctgtggatcagacgggtatacttacatatgggatagcagatctccgtccgctgtacaaagcttttacgttaacgatagtgatattaatgcacttcgttttttcaaagacgggatgtcgattgttgcaggaagtgacaatggtgcgataaatatgtatgatttaaggtcggactgttctattgctactttttctctttttcgaggttatgaagaacgtacccctacccctacttatatggcagctaacatggagtacaataccgcgcaatcgccacaaactttaaaatcaacaagctcaagctatctagacaaccaaggcgttgtttctttagattttagtgcatctggaagattgatgtactcatgctatacagacattggttgtgttgtgtgggatgtattaaaaggagagattgttggaaaattagaaggtcatggtggcagagtcactggtgtgcgctcgagtccagatgggttagctgtatgtacaggttcatgggactcaaccatgaaaatatggtctccaggttatcaatag

SEQ ID NO: 11 - protein, 422 aa, S. cerevisiae
MAHQMDSITYSNNVTQQYIQPQSLQDISAVEEEIQNKIEAARQESKQLHAQINKAKHKIQDASLFQMANKVTSLTKNKINLKPNIVLKGHNNKISDFRWSRDSKRILSASQDGFMLIWDSASGLKQNAIPLDSQWVLSCAISPSSTLVASAGLNNNCTIYRVSKENRVAQNVASIFKGHTCYISDIEFTDNAHILTASGDMTCALWDIPKAKRVREYSDHLGDVLALAIPEEPNLENSSNTFASCGSDGYTYIWDSRSPSAVQSFYVNDSDINALRFFKDGMSIVAGSDNGAINMYDLRSDCSIATFSLFRGYEERTPTPTYMAANMEYNTAQSPQTLKSTSSSYLDNQGVVSLDFSASGRLMYSCYTDIGCVVWDVLKGEIVGKLEGHGGRVTGVRSSPDGLAVCTGSWDSTMKIWSPGYQ

SEQ ID NO: 12 - DNA, 1269 bp, S. cerevisiae
atggcacatcagatggactcgataacgtattctaataatgtcacccaacagtatatacaaccacaaagtctacaggatatctctgcagtggaggaagaaattcaaaataaaatagaggccgccagacaagagagtaaacagcttcatgctcaaataaataaagcaaaacacaagatacaagatgcaagcttattccagatggccaacaaagttacttcgttgaccaaaaataagatcaacttaaagccaaatatcgtgttgaaaggccataataataaaatctcagattttcggtggagtcgagattcaaaacgtattttgagtgcaagtcaagatggctttatgcttatatgggacagtgcttcaggtttaaaacagaacgctattccattagattctcaatgggttctttcctgcgctatttcgccatcgagtactttggtagcaagcgcaggattaaacaataactgtaccatttatagagtttcgaaagaaaacagagtagcgcaaaacgttgcgtcaattttcaaaggacatacttgctatatttctgacattgaatttacagataacgcacatatattgacagcaagtggggatatgacatgtgccttgtgggatataccgaaagcaaagagggtgagaggatattctgaccatttaggtgatgttttggcattagctattcctgaagagccaaacttagaaaattcttcgaacacattcgctagctgtggatcagacgggtatacttacatatgggatagcagatctccgtccgctgtacaaagcttttacgttaacgatagtgatattaatgcacttcgttttttcaaagacgggatgtcgattgttgcaggaagtgacaatggtgcgataaatatgtatgatttaaggtcggactgttctattgctactttttctctttttcgaggttatgaagaacgtacccctacccctacttatatggcagctaacatggagtacaataccgcgcaatcgccacaaactttaaaatcaacaagctcaagctatctagacaaccaaggcgttgtttctttagattttagtgcatctggaagattgatgtactcatgctatacagacattggttgtgttgtgtgggatgtattaaaaggagagattgttggaaaattagaaggtcatggtggcagagtcactggtgtgcgctcgagtccagatgggttagctgtatgtacaggttcatgggactcaaccatgaaaatatggtctccaggttatcaatag

SEQ ID NO: 13 - protein, 422 aa, S. cerevisiae
MAHQMDSITYSNNVTQQYIQPQSLQDISAVEEEIQNKIEAARQESKQLHAQINKAKHKIQDASLFQMANKVTSLTKNKINLKPNIVLKGHNNKISDFRWSRDSKRILSASQDGFMLIWDSASGLKQNAIPLDSQWVLSCAISPSSTLVASAGLNNNCTIYRVSKENRVAQNVASIFKGHTCYISDIEFTDNAHILTASGDMTCALWDIPKAKRVRGYSDHLGDVLALAIPEEPNLENSSNTFASCGSDGYTYIWDSRSPSAVQSFYVNDSDINALRFFKDGMSIVAGSDNGAINMYDLRSDCSIATFSLFRGYEERTPTPTYMAANMEYNTAQSPQTLKSTSSSYLDNQGVVSLDFSASGRLMYSCYTDIGCVVWDVLKGEIVGKLEGHGGRVTGVRSSPDGLAVCTGSWDSTMKIWSPGYQ

SEQ ID NO: 14 - DNA, 1269 bp, S. cerevisiae
atggcacatcagatggactcgataacgtattctaataatgtcacccaacagtatatacaaccacaaagtctacaggatatctctgcagtggaggaagaaattcaaaataaaatagaggccgccagacaagagagtaaacagcttcatgctcaaataaataaagcaaaacacaagatacaagatgcaagcttattccagatggccaacaaagttacttcgttgaccaaaaataagatcaacttaaagccaaatatcgtgttgaaaggccataataataaaatctcagattttcggtggagtcgagattcaaaacgtattttgagtgcaagtcaagatggctttatgcttatatgggacagtgcttcaggtttaaaacagaacgctattccattagattctcaatgggttctttcctgcgctatttcgccatcgagtactttggtagcaagcgcaggattaaacaataactgtaccatttatagagtttcgaaagaaaacagagtagcgcaaaacgttgcgtcaattttcaaaggacatacttgctatatttctgacattgaatttacagataacgcacatatattgacagcaagtggggatatgacatgtgccttgtgggatataccgaaagcaaagagggtgagagaatattctgaccatttaggtgatgttttggcattagctattcctgaagagccaaacttagaaaattcttcgaacacattcgctagctgtggatcagacgggtatacttacatatgggatagcagatctccgtccgctgtacaaagcttttacgttaacgatagtgatattaatgcacttcgttttttcaaagacgggatgtcgattgttgcaggaagtgacaatggtgcgataaatatgtatgatttaaggtcggactgttctattgctactttttctctttttcgaggttatgaagaacgtacccctacccctacttatatggcagctaacatggagtacaataccgcgcaatcgccacaaactttaaaatcaacaagctcaagctatctagacaaccaaggcgctgtttctttagattttagtgcatctggaagattgatgtactcatgctatacagacattggttgtgttgtgtgggatgtattaaaaggagagattgttggaaaattagaaggtcatggtggcagagtcactggtgtgcgctcgagtccagatgggttagctgtatgtacaggttcatgggactcaaccatgaaaatatggtctccaggttatcaatag

SEQ ID NO: 15 - protein, 422 aa, S. cerevisiae
MAHQMDSITYSNNVTQQYIQPQSLQDISAVEEEIQNKIEAARQESKQLHAQINKAKHKIQDASLFQMANKVTSLTKNKINLKPNIVLKGHNNKISDFRWSRDSKRILSASQDGFMLIWDSASGLKQNAIPLDSQWVLSCAISPSSTLVASAGLNNNCTIYRVSKENRVAQNVASIFKGHTCYISDIEFTDNAHILTASGDMTCALWDIPKAKRVREYSDHLGDVLALAIPEEPNLENSSNTFASCGSDGYTYIWDSRSPSAVQSFYVNDSDINALRFFKDGMSIVAGSDNGAINMYDLRSDCSIATFSLFRGYEERTPTPTYMAANMEYNTAQSPQTLKSTSSSYLDNQGAVSLDFSASGRLMYSCYTDIGCVVWDVLKGEIVGKLEGHGGRVTGVRSSPDGLAVCTGSWDSTMKIWSPGYQ

SEQ ID NO: 16 - protein, 19 aa, S. cerevisiae
QYEFDVILSPELKVQMKTI

SEQ ID NO: 17 - protein, 19 aa, S. cerevisiae
QFKLPVIASDDLKVCKKSI

SEQ ID NO: 18 - protein, 19 aa, S. cerevisiae
QFKLPVIAFDDLKVCKKSI

SEQ ID NO: 19 - protein, 19 aa, S. cerevisiae
QFKLPVIASGDLKVCKKSI

SEQ ID NO: 20 - protein, 19 aa, S. cerevisiae
QFKLPVIAPDDLKVCKKSI